(19) United States
(12) Patent Application Publication
(10) Pub. No.: US 2018/0317761 A1
(43) Pub. Date: Nov. 8, 2018

(54) COLONIC POLYP REMOVAL

(71) Applicant: Salford Royal NHS Foundation Trust

(72) Inventors: Joanne Marie Thomas, Manchester (GB); Keith Patrick Heaton, Dorset (GB); Ian James Hardman, Dorset (GB); James Matthew Corden, Stockport (GB); Gordon Carlson, Manchester (GB)

(21) Appl. No.: 15/771,371

(22) PCT Filed: Oct. 21, 2016
(86) PCT No.: PCT/GB2016/053289

§ 371 Date: Apr. 26, 2018

(30) Foreign Application Priority Data
Oct. 26, 2015 (GB) 1518916.0

(51) Int. Cl. A61B 1/31; A61B 1/00; A61B 1/018; A61B 17/221

(52) U.S. Cl. CPC A61B 1/31; A61B 1/00078; A61B 1/00135; A61B 17/221; A61B 1/00154; A61B 1/0014; A61B 1/018

(57) ABSTRACT

Herein described is an instrument guide for use with a colonoscope, the guide being adapted to be mounted to the colonoscope. The guide defines a channel along which an instrument can be passed during use. Also described is a method of removing a polyp from a colon including inserting a colonoscope into the colon, inserting an instrument guide into the colon, mounting the guide to the colonoscope, inserting an excision instrument, excising a polyp and withdrawing the excised polyp from the colon.

### FIELD OF THE INVENTION

The present invention relates to an instrument guide for use with a colonoscope. The invention further relates to a method of removing a polyp from the colon and a kit of parts comprising a colonoscope and guide, or guide and applicator for the guide.

### BACKGROUND OF THE INVENTION

Colorectal cancer is the third most common cancer in the UK with approximately 35,000 new cases diagnosed each year. The majority of these colorectal cancers are caused by colonic polyps, small growths on the inner lining of the colon, which are extremely common, especially in the over 60s. Polyps are often harmless but can progress from benign to malignant growths, if left untreated.

Regular bowel cancer screening has been shown to reduce the risk of dying from the disease by 16%. Screening in the UK is offered to men and women aged 60 to 69 and involves testing for blood in the faeces. If an abnormal result is obtained, the patient is offered a colonoscopy which involves passing a colonoscope through the rectum into the colon, to investigate the lining of the bowel.

Colonoscopes are highly specialised instruments which generally consist of a housing and a flexible insertion tube with a distal camera and light. The housing typically includes an eyepiece and controls for moving the distal tip of the insertion tube. Colonoscopes also have channels within the insertion tube for suction, water and air delivery, and the insertion and removal of instruments. However, the size of these channels is limited due to the size constraints placed on the colonoscope.

During operation, the colonoscope is passed into the colon and advanced along the colon by the surgeon or clinician, who uses the controls on the housing to direct the colonoscope around the tight bends of the colon. This process is time consuming and requires experience and dexterity on the part of the surgeon or clinician. If polyps are identified they can be removed during the procedure.

Small polyps, for instance those which are less than around 7 mm in diameter, can often be relatively easily removed from the bowel. Techniques for removal of small polyps include cold or hot snaring. The snares are passed through an instrument channel of the colonoscope and opened over the polyp. Closing the snare cuts the polyp from the bowel lining. Hot snaring involves the utilisation of electro-cautery to cut through the polyp tissue by connecting the snare to a diathermy machine. The polyps are subsequently extracted from the bowel via a suction channel of the colonoscope. However, removal of larger polyps (for example polyps with a size greater than 1 cm), which are often more advanced, pose more of a problem.

The current method for removing larger polyps includes snaring the polyp to cut the polyp from the bowel lining and subsequently capturing the polyp in an expanding basket known as a Roth net. The snare and the Roth net are advanced through the instrument channel in the colonoscope to the location of the polyp. However, once the polyp is contained within the Roth net, the polyp cannot be removed through the colonoscope channel because the polyp is too large to pass through the channel. As such, the only way to remove the polyp is to retract the colonoscope from the patient, dragging the Roth net and polyp behind the colonoscope. As an alternative to a Roth net, the polyp can be suctioned onto the tip of the colonoscope and extracted by removal of the colonoscope.

It is important to identify the location of the or each polyp in the bowel. Should subsequent surgery be required, for example, it is important to be able to ascertain the location from which the/each polyp was removed. This is difficult if multiple polyps are captured in a single Roth net. Therefore, when multiple polyps are present (in 10-30% of cases) the colonoscope must be inserted and removed from the bowel multiple times. Not only does this result in discomfort for the patient but, in addition, only a certain number of re-insertions will be tolerated and there is therefore an increased likelihood that a malignant polyp will go undetected. Multiple re-insertions also increase the time taken to screen each patient, placing further strain on the resources of the healthcare system.

Most colonoscopies are performed without complication. However, occasionally insertion of the colonoscope may cause damage to the colon lining which can, in certain instances, result in bleeding, infection and/or perforation of the bowel. The likelihood of such complications is increased when multiple re-insertions of the colonoscope are required. There is therefore a desire to develop a colonoscopy approach which avoids the problems associated with multiple re-insertions of the colonoscope.

It is an object of the present invention to obviate or mitigate one or more of the abovementioned disadvantages, and/or to provide an improved method and/or apparatus for removing polyps from the bowel.

### SUMMARY OF THE INVENTION

According to a first aspect of the invention there is provided an instrument guide for use with a colonoscope, the guide being adapted to be mounted to the colonoscope; and wherein the guide defines a channel along which an instrument can be passed during use.

The instrument passed down the channel may be the colonoscope. Alternatively, the instrument may be an instrument for excising tissue from the wall of the colon, for example a snare for hot or cold snaring, or an instrument for capturing tissue (such as a Roth net. The person skilled in the art will appreciate that the instrument may be any surgical instrument used with a colonoscope.

Reference to the guide being mounted to the colonoscope is intended to include the guide and colonoscope being attached to one another (for instance using attachment features as discussed below), the colonoscope being received within the guide (for instance the guide substantially surrounding the colonoscope), or any other suitable manner in which the guide and colonoscope engage one another during use.

The channel of the present invention may be open-sided, for example generally U-shaped in cross section, or in an alternative, may be closed-sided, for example generally tubular in shape.

The channel may be configured such that the instrument can be passed generally alongside the colonoscope when the guide is mounted to the colonoscope.

As discussed above, in a conventional procedure, a surgical instrument (for excising and/or capturing a polyp, for example) is generally passed through a port in the colonoscope. To remove a polyp greater than a certain size from the colon (for example greater than 7 mm), the instrument and polyp cannot simply be retracted through the port of the colonoscope. As such, the colonoscope itself (and therefore the instrument and polyp) must be withdrawn from the colon and subsequently re-inserted (if further surgery/inspection is to be performed), increasing the risk of damage to the lining of the colon and/or discomfort to the patient. The present invention may be advantageous in that an instrument can be passed repeatedly through the channel of the guide during a procedure. This may allow the instrument to be inserted repeatedly into the colon and subsequently removed, with a polyp for example, with the guide acting as a barrier between the lining of the colon and the instrument. Such an approach may reduce the risk of damage to the colon and/or discomfort to the patient.

The guide may comprise a longitudinal array of generally annular or C-shaped ribs. Such ribs may be substantially evenly spaced along the length of the guide. Where the ribs are generally C-shaped, the ribs may be configured to allow mounting of the guide to a colonoscope. For instance, the ribs may be sized such that they clip onto the colonoscope and retain the guide in place on the colonoscope. The ribs may also allow the guide to be retained in place on the colonoscope whilst the guide is fed around the tight bends of the colon.

The guide may be generally in the form of a hollow tubular structure. The guide may, when in use, act as a sheath to the colonoscope, by surrounding (completely or partially) the colonoscope. In such embodiments, the channel may be formed due to the internal diameter of the guide being greater than the external diameter of the colonoscope. In an alternative, the hollow formed by the tubular structure may form the channel, at which point the instrument passed through the channel may be the colonoscope (as well as, or instead of any other surgical instrument). In another alternative, the guide may comprise a two channel structure, the first channel being formed by a hollow tubular structure in which the colonoscope can be placed and the second channel running generally longitudinally along the first channel. A surgical instrument may, in use, be accommodated in said second channel.

The tubular structure may, in embodiments, comprise a split running generally along the tubular structure. The presence of a split may allow the guide to be fitted to the colonoscope whilst the colonoscope is in use, i.e. while the distal end of the colonoscope is within a patient, since the split allows the guide to be fitted to the colonoscope over the handle of the colonoscope. The ability to fit the guide to the colonoscope whilst the colonoscope is within a patient enables the guide to form a lining in the bowel, following complex navigation of the bowel by the colonoscope. Once the guide is in place, the colonoscope can then be removed and re-inserted into the bowel through the lining formed by the guide without risking damage to the lining of the colon.

In embodiments, the split is held substantially closed by fastening means when in use, for example when mounted to the colonoscope. In embodiments, the fastening means may be a zip-lock fastener. Alternatively, or in addition, the tubular structure may be resiliently deformable. Such resilient deformability may result in the split being held substantially closed (or as closed as is possible when the colonoscope is located within the guide). As mentioned above, the colonoscope has to navigate the tight bends of the colon. By the split being held substantially or partially closed in use, the possibility of the guide separating or detaching from the colonoscope, whilst the guide is passed along these tight bends for example, may be reduced.

In embodiments, the tubular structure may comprise corrugations. Alternatively, or in addition, the tubular structure may comprise fenestrations or apertures. Such corrugations and/or fenestrations or apertures may enable the guide to traverse the tight bends of the colon more easily, without being removed from a colonoscope, for example.

The guide may be deformable in a radial direction. Such radial deformability may allow the guide to stretch to allow passage of the instrument (and/or a polyp) along the channel. The deformability of the guide may also aid mounting of the guide to the colonoscope (for instance where the colonoscope is introduced longitudinally into the guide, the guide may stretch to allow easy insertion and the restorative force from stretching the guide may cause the guide to remain in position on the colonoscope).

In embodiments of the invention the guide may further comprise a stiffening element positioned to stiffen the guide in a longitudinal direction. The stiffening element may be one or more elongate struts, or a patch or layer of relatively rigid material, for example. The presence of such stiffening elements may aid transmission of a pushing force of the surgeon or clinician along the whole length of the guide (by providing axial rigidity to the guide) and/or reduce or prevent axial compression of the guide on insertion of the guide into the colon. Such stiffening elements may also be useful in aiding fitment/securement of the guide to the colonoscope. For instance, the stiffening elements may act as handles to allow easy manipulation of the guide, and/or may allow the guide to be moved longitudinally relative to the colonoscope without “bunching up”.

In embodiments of the invention, the guide may further comprise an attachment feature for coupling of the guide to the colonoscope. The attachment feature may be configured for coupling the guide to a distal end of the colonoscope. By the term “distal end” of the colonoscope we refer to the end that is furthest from the handle of the colonoscope, i.e. the end of the colonoscope that is inserted into the colon. Such attachment features may be particularly useful when the colonoscope and guide are inserted into the colon substantially simultaneously. For example, if the guide and colonoscope are coupled at a distal end, this will reduce the likelihood of the guide being axially compressed or “bunching-up” when the guide and colonoscope are inserted into the colon. In this way, the guide may be pulled up the colon by the colonoscope.

Suitable attachment features will be appreciated by the person skilled in the art but may be interlocking protrusions and recesses, a magnetic coupling or a screw attachment, for example.

In embodiments, the attachment feature may be a remotely actuable coupling mechanism. The ability to actuate the attachment feature remotely may allow the coupling of the guide to the colonoscope to be disengaged whilst the colonoscope and guide are inside the patient. This may allow the colonoscope or the guide to be removed from the colon independently of the other. For example, the guide could be withdrawn and subsequently re-inserted into the colon without having to withdraw and subsequently re-insert the colonoscope. By re-inserting only some of the instruments used during a colonoscopy, the risk of injury to the lining of the colon and/or discomfort for the patient may be reduced.

The guide of the present invention may be fabricated from any suitable material. In embodiments of the invention the guide may be fabricated from a thermoplastic material such as polyoxymethylene (otherwise known as acetal), polyurethane or nylon. For the avoidance of doubt, it will be appreciated that the guide may be fabricated from more than one material, for example, it may be fabricated from a polymer within which metallic fibres or other reinforcements are located. As another example, the guide may be fabricated from an array of nylon ribs within a polyurethane matrix. Useful properties of materials used for fabrication of the guide of the present invention may include low coefficient of friction (to allow easy passage of the guide within the colon), a suitable balance between rigidity and elasticity (the guide being sufficiently rigid to enable a pushing force to be transmitted along the length of the guide and sufficiently elastic to allow the guide to pass around the tight bends in the colon and accommodate large polyps, for example), and biocompatibility.

In a second aspect of the invention there is provided a kit of parts comprising a colonoscope and a guide according to the first aspect of the invention.

In a third aspect of the invention there is provided a kit of parts comprising a guide according to the first aspect of the present invention and an applicator for applying the guide to a colonoscope. In embodiments, the kit may further comprise a colonoscope.

In yet a further aspect of the invention there is provided an applicator (for example the applicator of the third aspect of the invention).

The applicator may comprise a guide engaging portion for engaging a guide according to the first aspect of the invention. The guide engaging portion may be generally arcuate in shape. In embodiments, the arcuate portion tapers away from the colonoscope. Such a shape allows a guide having a split to be applied to the colonoscope using the applicator, when the distal end of the colonoscope is within the colon. The arcuate portion may increase the width of the split to allow the guide to be inserted over the colonoscope.

In embodiments, the applicator may further comprise a guide closing portion arranged to urge the sides of the split guide towards one another to close or partially close the split, when the guide has been inserted over the colonoscope.

The applicator may further comprise a colonoscope engaging portion. The colonoscope engaging portion may, for example, comprise an inner surface of complementary shape to the outer surface of the colonoscope, for example a body with a cylindrical bore. The inner surface may be configured to provide an interference fit between the applicator and the colonoscope. In embodiments, the colonoscope engaging portion may be provided with a handle portion. Such a handle may be used to hold the applicator in position relative to the colonoscope as the guide is applied to the colonoscope. In embodiments, the handle will be provided with a textured surface, for example a ribbed or knurled surface. Such a surface will provide sufficient friction when gripped by a surgeon or clinician wearing surgical gloves (which may be coated with lubricant) during operation.

In embodiments, the colonoscope engaging portion may comprise a clamp which can be deployed to secure the applicator in position relative to the colonoscope. The presence of such a clamp allows the applicator to be retained in position without exertion of a clamping force by the user. This can be advantageous as it allows a single user to hold the colonoscope/applicator in one hand and apply a force to the guide with the other hand to apply the guide to the colonoscope.

The applicator can be fabricated of any suitable material, but it will be appreciated that the material should be sufficiently flexible that the colonoscope engaging portion of the applicator can be deformed to be fitted to the colonoscope.

In a further aspect of the present invention there is provided a method for removing a polyp from a colon, the method comprising:
inserting a colonoscope into the colon;
inserting a guide according to the first aspect of the invention into the colon;
mounting the guide to the colonoscope;
inserting an excision instrument for excising a polyp from a wall of the colon;
excising the polyp using said excision instrument; and
withdrawing the excised polyp from the colon.

As will be appreciated by a person skilled in the art, the order in which the above steps are recited does not necessarily correspond to the order in which the steps must be performed. For example, the guide may be mounted to the colonoscope before the colonoscope is inserted into the colon (at which point the colonoscope and guide may then be inserted into the colon simultaneously).

Withdrawal of the excised polyp may take place by withdrawing the polyp down the channel of the guide. In such embodiments, the guide and colonoscope may remain within the colon whilst the excised polyp is withdrawn from the colon. By reducing or removing the need for withdrawal and re-insertion of the colonoscope into the colon during the procedure, damage to the lining of the colon and/or discomfort to the patient may be minimised or reduced.

In embodiments, the method may further comprise inserting a withdrawal instrument prior to the step of withdrawing the excised polyp from the colon, wherein the step of withdrawing the excised polyp from the colon is achieved using the withdrawal instrument. In embodiments, the withdrawal instrument may have a Roth net, for example.

The step of inserting a colonoscope into the colon may take place prior to the step of inserting the guide into the colon. In this case, the colonoscope may act as a “track” along which the guide is passed. In such embodiments, the guide may comprise a split along its length to allow the guide to be mounted to the colonoscope over the handle of the colonoscope when a distal end of the colonoscope is inside the patient.

Alternatively, the steps of inserting the colonoscope into the colon and inserting the guide into the colon may be performed substantially simultaneously.

In embodiments, the step of mounting the guide to the colonoscope comprises connecting the guide to the colonoscope, for example by way of attachment features such as interlocking protrusions and recesses, a magnetic coupling or a screw attachment, for example. In such embodiments, the step of connecting the guide to the colonoscope may take place prior to the steps of inserting the colonoscope into the colon and inserting the guide into the colon.

The method of this aspect of the invention may be used to remove multiple polyps from the colon. In such embodiments, the method may further comprise excising a further polyp using said excision instrument; and withdrawing the excised further polyp from the colon, optionally using a withdrawal instrument. Excision of the further polyp may take place after withdrawing the initial polyp from the colon, or before then (at which point the two polyps may or may not be removed together).

In embodiments of the invention, the method may further comprise withdrawing the guide from the colon. The step of withdrawing the guide from the colon may take place substantially simultaneously with the step of withdrawing the excised polyp from the colon, or after said step or before said step.

In an alternative, the method may further comprise withdrawing the colonoscope from the colon. The step of withdrawing the colonoscope from the colon may take place substantially simultaneously with the step of withdrawing the excised polyp from the colon, after said step or before said step.

### DETAILED DESCRIPTION OF THE INVENTION

The present invention will now be described with reference to the following non-limiting examples and figures, which show:

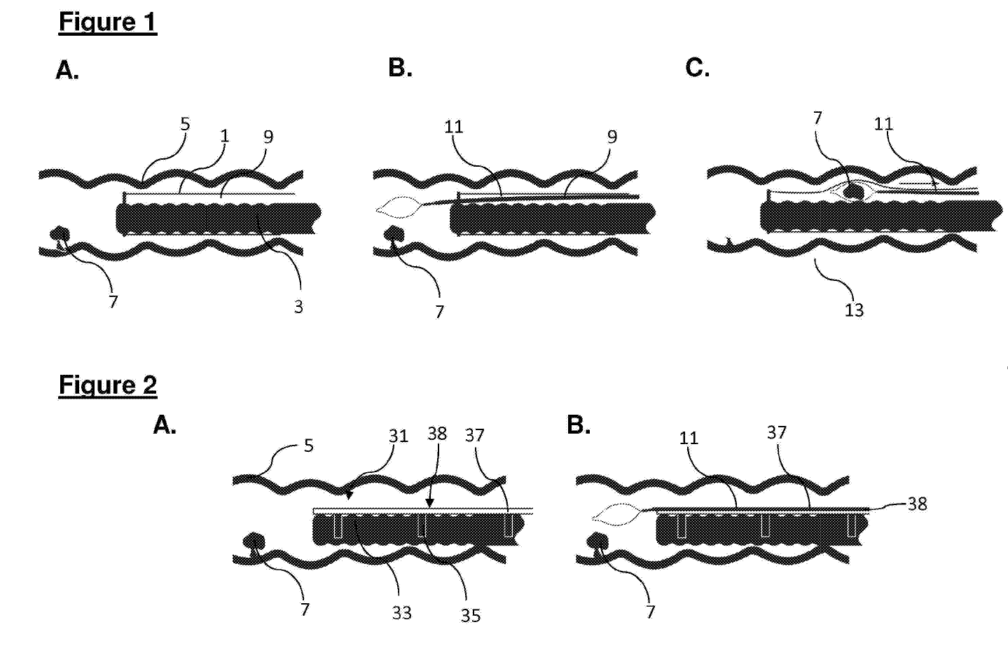

FIG. 1: Schematic representations of a guide according to a first embodiment of the present invention mounted to a colonoscope;

FIG. 2: Schematic representations of a guide according to a second embodiment of the present invention mounted to a colonoscope;

FIG. 3: Schematic representations of a guide according to a third embodiment of the present invention mounted to a colonoscope;

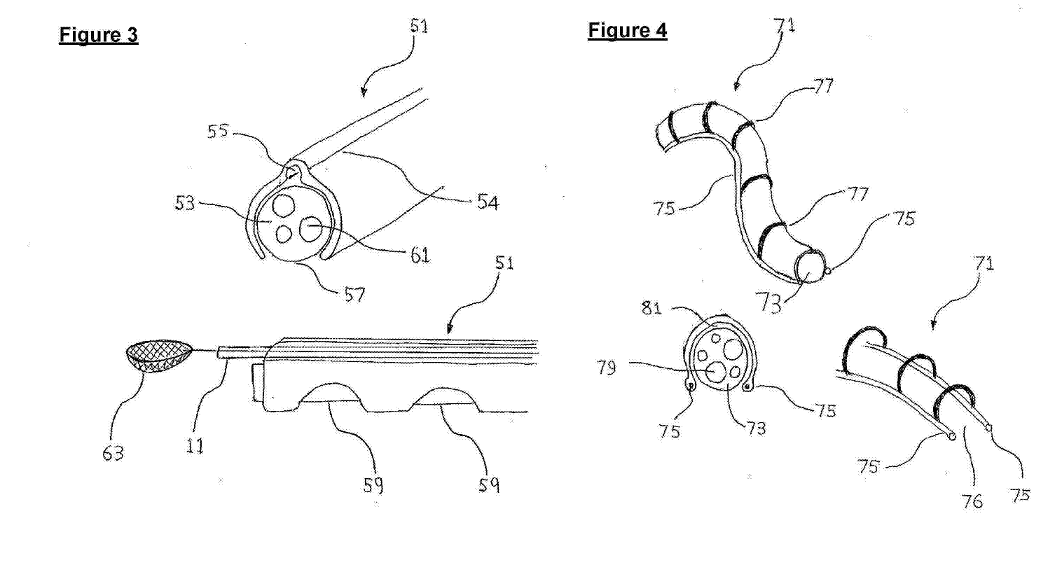

FIG. 4: Schematic representations of a guide according to a fourth embodiment of the present invention mounted to a colonoscope;

FIG. 5: Schematic representations of a guide according to a fifth embodiment of the present invention mounted to a colonoscope;

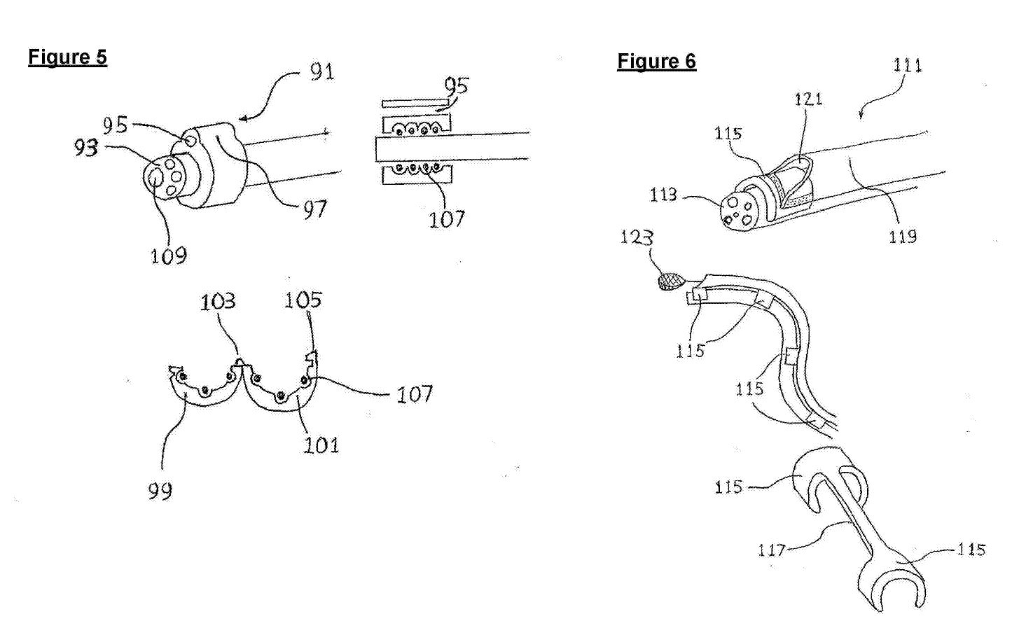

FIG. 6: Schematic representations of a guide according to a sixth embodiment of the present invention mounted to a colonoscope;

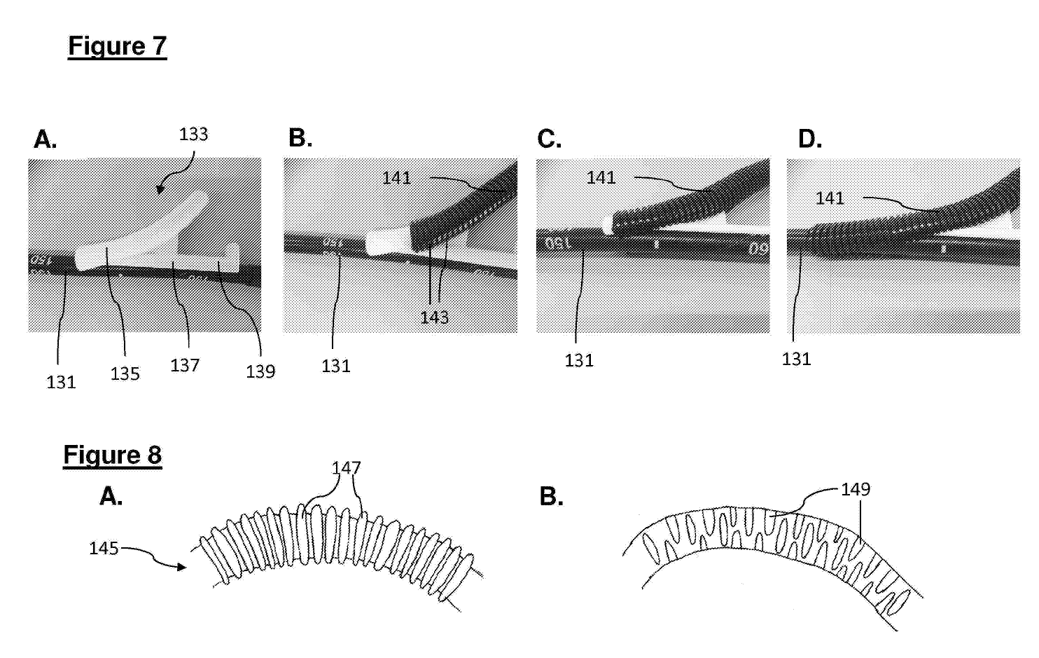

FIG. 7: Side views showing application of a guide according to an embodiment of the present invention to a colonoscope;

FIG. 8: Side views showing two guides according to embodiments the present invention;

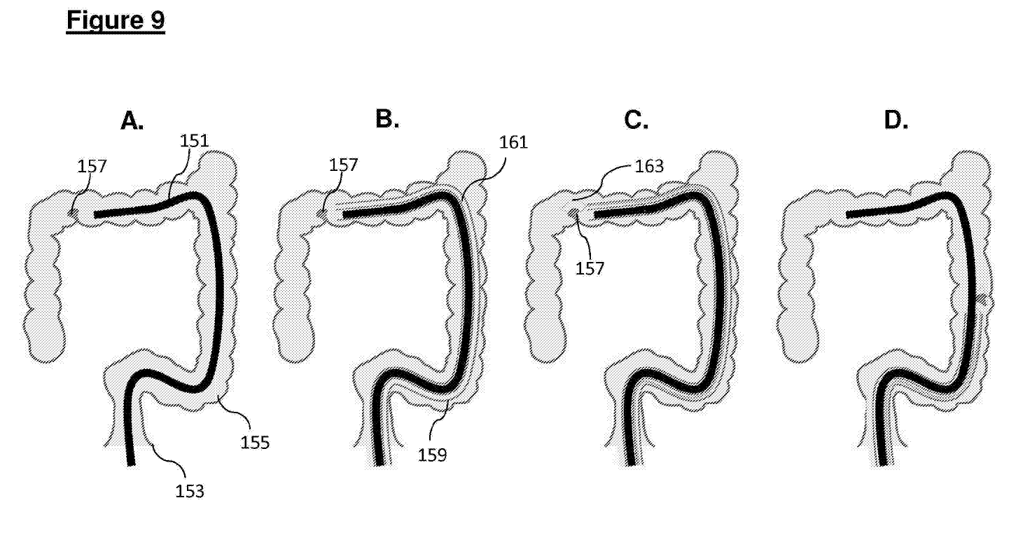

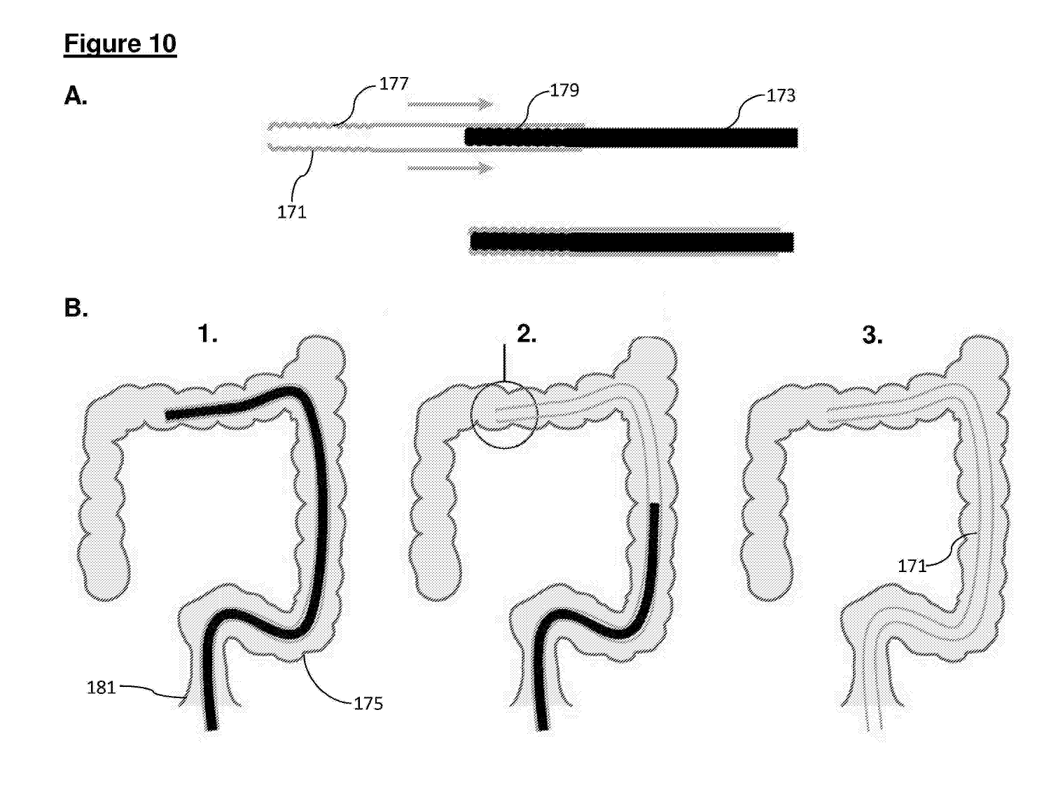

FIG. 9: Schematic representations of a method of removing a polyp from the colon using a colonoscope and a guide according to an embodiment of the present invention; and

FIG. 10: Schematic representations of a method of removing a polyp from the colon using a colonoscope and a guide according to an embodiment of the present invention.

Referring to FIG. 1 there is depicted a guide 1 according to a first embodiment of the present invention. The guide 1 is mounted to a generally tubular colonoscope 3 (the distal end of which is visible in this figure) located within a colon 5 of a patient (not shown). In this case, the guide 1 is a sheath which is generally in the shape of an elongate tube which surrounds the colonoscope 3. In this embodiment, an inner diameter of the guide 1 has a greater diameter than an outer diameter of the colonoscope 3. This difference in diameter results in a generally crescent-shaped channel 9 being defined between the colonoscope 3 and the guide 1.

Although this embodiment of the invention is described in relation to the channel 9 being the crescent-shaped cavity that is formed cooperatively by the colonoscope 3 and guide 1, it may equally be considered that the entire internal hollow of the guide 1 forms the channel (at which point the channel would be generally circular in cross section).

The colonoscope 3 is used to locate a polyp 7 within the colon 5. The guide 1 may either be positioned on the colonoscope 3 prior to insertion of the colonoscope 3 into the colon or, in an alternative, the guide 1 may be advanced along the colonoscope 3 when the colonoscope 3 is positioned within the colon, the colonoscope 3 thereby acting as a track along which the guide 1 is passed. When a polyp 7 is located within the colon 5, a surgical instrument 11 is passed along the channel 9 alongside the colonoscope 3 until the instrument 11 reaches the site of the polyp 7 (see FIG. 1B). In FIG. 1B, the instrument 11 is an excision instrument, for example a hot or cold snare, which can be used to excise the polyp 7 from the lining of the colon 5. An additional instrument 11, for example an instrument having a Roth net, is then used to capture the polyp 7. The captured polyp 7 can then be withdrawn from the colon 5 by withdrawing the instrument 11 down the channel 9 (see FIG. 10).

The colonoscope 3 can then be used to locate additional polyps 7. If an additional polyp 7 is located, surgical instruments 11 can once again be passed along the channel 9 until they 11 reach the site of the polyp 7. The polyp 7 can then be removed and withdrawn from the colon 5 in the same way as described above. The presence of the guide 1 and channel 9 allows multiple polyps 7 to be removed from the colon 5 without having to withdraw and insert the colonoscope 3 multiple times, which may result in damage to the colon lining and/or discomfort for the patient.

Referring now to FIG. 2 there is depicted a guide 31 according to a second embodiment of the present invention. The guide 31 is mounted to a generally tubular colonoscope 33 located within a colon 5 of a patient (not shown). In this embodiment, the guide 31 comprises an array of C-shaped ribs 35 which can be used to snap-fit the guide 31 to the colonoscope 33. The ribs 35 are distributed along the axial length of the guide 31. In this case, the ribs 35 are spaced substantially regularly along the length of the guide 31. However, in other embodiments the distribution of the ribs 35 may vary along the length of the guide 31. The ribs 35 provide a degree of rigidity to the guide 31 as a whole, but the existence of spacing between the ribs 35 allows the guide 31 to bend (for instance with the colonoscope 33).

The guide 31 further comprises an elongate channel 37 defined within a spine 38 which connects the ribs 35 to one another. The channel 37 is configured to receive a surgical instrument 11 such that the instrument 11 can be passed to the site of the polyp 7 (see FIG. 2B).

As with the example of FIG. 1, the colonoscope 33 of FIG. 2 is used to locate a polyp 7 within the colon 5. The guide 31 may be positioned on the colonoscope 33 before insertion of the colonoscope 33 into the colon 5 or alternatively, the guide 31 may be advanced along the colonoscope 3 once a distal end of the colonoscope is within the colon 5, the colonoscope 3 thereby acting as a track along which the guide 31 is passed. The guide 31 is sufficiently axially rigid that the guide 31 does not bunch up as it is inserted into the colon. The ribs 35 prevent the guide 31 from becoming detached from the colonoscope 33 whilst the guide 31 is advanced along the colon. When a polyp 7 is located within the colon 5 a surgical instrument 11 is passed along the channel 37 until the instrument 11 reaches the site of the polyp 7 (see FIG. 2B). In FIG. 2B, the instrument 11 is an excision instrument which can be used to excise the polyp 7 from the colon 5. An additional instrument 11, for example an instrument having a Roth net, is then used to capture the polyp 7. The captured polyp 7 can then be withdrawn from the colon 5 by withdrawing the instrument 11 down the channel 37.

If an additional polyp 7 is located, surgical instruments 11 can once again be passed along the channel 37 until they reach the site of the polyp 7. The polyp 7 can then be removed and withdrawn from the colon 5 in the same way as described above. The presence of the guide 31 and channel 37 allows multiple polyps 7 to be removed from the colon 5 without having to withdraw and insert the colonoscope 33 multiple times.

In the procedure shown in FIG. 2, the guide 31 is retained in its mounted position on the colonoscope 33 whilst the surgical instrument 11 is withdrawn from the colon 5. In an alternative, the instrument 11 and guide 31 could be inserted and withdrawn from the colon 5 substantially simultaneously by sliding the guide 31 along the colonoscope 33.

Referring now to FIG. 3 there is depicted a guide 51 according to a third embodiment of the invention. As with the embodiments of FIGS. 1 and 2, the guide 51 of FIG. 3 is mounted to a generally tubular colonoscope 53. In this case, the guide 51 is a sheath which is generally in the shape of an elongate tube which surrounds the colonoscope 53. In this case, the guide 51 has a ridge 54 which runs generally longitudinally along the guide 51. The ridge 54 defines a channel 55 which can accommodate a surgical tool 11.

As with the first embodiment, the third embodiment is described in terms of the channel 55 being formed cooperatively between the guide 51 and colonoscope 53. However, it may equally be considered that the entire internal hollow of the guide 51 forms the channel.

In this embodiment the guide 51 has a split 57 which runs down the length of the guide 51. The presence of the split 57 allows the guide 51 to be mounted onto the colonoscope 53 after the colonoscope 53 has been inserted into the colon without having to run the guide 51 down the length of the colonoscope 53. This is described in more detail below.

The guide 51 further comprises notches 59 along the length of the guide 51. Such notches 59 aid bending of the guide 51 downwards from the perspective of FIG. 3 by reducing the amount of material on the side of the guide 51 which must be compressed. Such notches 59 also allow the guide 51 to remain on the colonoscope 53 when navigating tight bends in the colon and allow axial pushing forces to be transmitted along the guide 51.

In use, the colonoscope 53 is inserted into the colon (not shown). The colonoscope 53 is used to locate a polyp (not shown) within the colon. The guide 51 may either be positioned on the colonoscope 53 prior to insertion of the colonoscope 53 into the colon or, in an alternative, the guide 51 may be advanced along the colonoscope 53 when the colonoscope 53 is positioned within the colon, the colonoscope 53 thereby acting as a track along which the guide 51 is passed. When a polyp is located within the colon a surgical instrument 11 is passed along the channel 55 until the instrument 11 reaches the site of the polyp. An excision instrument (not shown) fed through a port 61 in the colonoscope 53 is used to excise the polyp from the colon. Instrument 11 is then used to capture the polyp by way of a net 63, for example a Roth net. The captured polyp can then be withdrawn from the colon by withdrawing the instrument 11 down the channel 55.

If an additional polyp is located the surgical instrument 11 can once again be passed along the channel 55 until the instrument 11 reaches the site of the polyp. The polyp is removed and withdrawn from the colon in the same way as described above. The presence of the guide 51 and channel 55 allows multiple polyps to be removed from the colon without having to withdraw and re-insert the colonoscope 53 into the colon multiple times.

Referring now to FIG. 4 there is depicted a guide 71 according to a fourth embodiment of the invention. As with the embodiments of FIGS. 1-3, the guide 71 of FIG. 4 is mounted to a generally tubular colonoscope 73. Similar to the guide 51 of FIG. 3, the guide 71 of FIG. 4 is a sheath which is generally in the shape of an elongate tube which surrounds the colonoscope 73 during use. The guide 71 of this embodiment is elastically deformable which allows the guide 71 to expand radially to accommodate a surgical instrument or polyp (not shown) running generally alongside the colonoscope 73.

The guide 71 has a split 76 which in this case runs generally along the length of the guide 71. The guide 71 further comprises two stiffening elements 75 positioned to stiffen the guide 71 in the longitudinal direction. In this embodiment, the stiffening elements 75 run along either side of the split 76. The stiffening elements 75 resist axial compression of the guide 71 (e.g. prevent the guide “bunching up” when inserted into the colon), aid fitment of the guide 71 to the colonoscope 73 and allow pushing forces to be transmitted along the length of the colonoscope 73.

The guide 71 further comprises ribs 77 which, in this case, are distributed along the axial length of the guide 71. In this case, the ribs 77 are spaced substantially regularly along the length of the guide 71. However, in other embodiments the distribution of the ribs 77 may vary along the length of the guide 71. Each rib 77 in generally C-shaped and runs generally circumferentially around the guide 71, between the two stiffening elements 75.

In this embodiment, the colonoscope 73 can be inserted into the guide 71 by forcing the colonoscope 73 through the split 76 in the guide 71. In this example, the guide 71 is elastically deformable, for example it is elastically deformable in the circumferential and radial directions. The guide 71 being deformable in the circumferential direction allows the stiffening elements 75 to be moved apart from one another so as to increase the circumferential width of the split 76 and thereby to allow the guide 71 to be inserted onto the colonoscope 73. The restorative force from this deformation acts to urge the stiffening elements 75 towards one another to reduce the circumferential width of the split 76, for instance to close (or partially close) the split 76 behind the colonoscope 73 once the guide 71 has been inserted onto the colonoscope 73. This restorative force also helps to secure the guide 71 around the colonoscope 73. The guide 71 being deformable in the radial direction allows the guide 71 to expand to accommodate a surgical instrument or polyp. In an alternative, an additional lumen could be present in the guide 71 to accommodate the surgical instrument or polyp.

In use, the colonoscope 73 is used to locate a polyp (not shown) within the colon. When a large polyp is located within the colon, which cannot be removed through a port 79 in the colonoscope 73, guide 71 is fitted to the colonoscope 73 and run along the length of the colonoscope 73 to the location of the polyp. In this way, the colonoscope 73 acts as a “track” along which the guide 71 can pass. A surgical instrument (not shown) is passed along a channel 81 formed between the colonoscope 73 and the guide 71. The instrument may be an excision instrument which can be used to excise the polyp from the colon. The instrument is then used to capture the polyp by way of a Roth net for example. The captured polyp can then be withdrawn from the colon by withdrawing the instrument through channel 81.

If an additional polyp is located, the surgical instrument can once again be passed along the channel 81 until the instrument reaches the site of the polyp. The polyp is removed and withdrawn from the colon in the same way as described above. The presence of the guide 71 and the channel 81 allows multiple polyps to be removed from the colon without having to withdraw and re-insert the colonoscope 73 into the colon multiple times.

It will be appreciated, that although in the above description the guide 71 is fitted to the colonoscope after the colonoscope 73 has been inserted into the colon, the colonoscope 73 and guide 71 could be inserted into the colon substantially simultaneously. In such an embodiment, the guide 71 may be attached to the colonoscope 73 at a distal end of the colonoscope 73 (for instance using restorative force from deformation of the guide, as discussed above, and/or using a different mechanism) to allow the guide 70 to be pulled along the colon by the colonoscope 73 and to prevent any “bunching-up” of the guide 71 on insertion into the colon.

Referring now to FIG. 5 there is depicted a guide 91 according to a fifth embodiment of the invention. As with the embodiments of FIGS. 1-4, the guide 91 of FIG. 5 is mounted to a generally tubular colonoscope 93. In this case, the guide 91 is a substantially annular clamp which circumferentially surrounds the distal end of the colonoscope 93. The guide 91 has a channel 95 defined by a ridge 97 which runs generally longitudinally along the guide 91. In this case the guide 91 is formed from two portions 99 and 101 connected to one another by a hinge 103. When the guide 91 is to be attached to the colonoscope 93 the two portions 99, 101 are hinged to an open position. The guide 91 is then secured around the colonoscope 93 by hinging the portions 99, 101 to a closed position in which the clamp circumferentially surrounds the colonoscope 93. The two portions 99, 101 are secured in the closed position using a clasp 105. In an alternative embodiment, the two portions 99 and 101 may not be hingedly connected and instead may connect to one another via a snap fit connection feature, for example.

The guide 91 further includes a plurality of bearings 107, in this case ball bearings, positioned on a radially inner (colonoscope facing) side of the guide 91. When the guide 91 is mounted to the colonoscope 93 the ball bearings 107 allow the guide 91 to move smoothly up and down the length of the colonoscope 93.

Similar to the examples of FIGS. 1-4, in use the colonoscope 93 is used to locate a polyp within the colon. If a large polyp is located, which cannot be withdrawn through a port 109 in the colonoscope 93, the guide 91 is affixed to the colonoscope 93 and run up the length of the colonoscope 93 using ball bearings 107. A surgical instrument may be located in channel 95, and thereby positioned adjacent the polyp. An instrument is moved out of the channel 95 towards the polyp and, when the surgical instrument is an excision instrument, is used to excise the polyp from the colon. The instrument is then used to capture the polyp by way of a Roth net, for example. The captured polyp can then be withdrawn from the colon by running the guide 91 down the length of the colonoscope 93.

If an additional polyp is located, the guide 91 can once again be run along the length of the colonoscope 93 until the guide 91 reaches the site of the polyp. Once again, the instrument located within the channel 95 can be used to excise and withdraw the polyp from the colon in the same way as described above. The presence of the guide 91 and channel 95 allow multiple polyps to be removed from the colon without having to withdraw and re-insert the colonoscope 93 into the colon multiple times.

Referring now to FIG. 6 there is depicted a guide 111 according to a sixth embodiment of the invention. As with the embodiments of FIGS. 1-5, the guide 111 of FIG. 6 is mounted to a generally tubular colonoscope 113. In this case, the guide 111 comprises a longitudinal array of generally C-shaped ribs 115. The ribs 115 are connected to one another by a generally longitudinal spine 117. The spine 117 is positioned to stiffen the guide 111 in a longitudinal direction. The spine 117 resists axial compression of the guide 111 (e.g. prevents the guide “bunching up” when inserted into the colon).

The ribs 115 are resiliently deformable to enable fitment to the colonoscope 113. The ends of the ‘C’ of each rib 115 can be moved apart from one another to allow passage of the colonoscope 113 into the guide 111.

An elongate sheath 119 is attached to the ribs 115, in this case by RF welding. The sheath 119 forms a channel 121 along which a surgical instrument 123 and/or polyp (not shown) can be passed. In this case, the sheath 119 extends circumferentially over around half of the colonoscope 113, leaving the other circumferential half of the colonoscope 113 exposed (with the exception of the portions thereof covered by the ribs 115).

The guide 111 of the sixth embodiment of the invention can be used in a manner similar to that described above in relation to previous embodiments.

Referring now to FIG. 7A there is depicted a colonoscope 131 fitted with an applicator 133 for fitting a guide 141 according to an embodiment of the present invention to the colonoscope 131. The applicator 133 has a generally arcuate portion 135 for engaging a guide 141 according to an embodiment of the present invention (as shown in FIG. 7B). The applicator 133 also has a colonoscope-engaging section 137 which aligns the applicator 133 with the colonoscope 131. An end of the colonoscope engaging section 137 distal from arcuate portion 135 defines a handle 139 which can be used to hold the applicator 133 in position as the guide 141 is applied to the colonoscope 131. The handle 139 may have texturing or knurling, so as to provide sufficient friction when gripped by a surgeon or clinician wearing surgical gloves during operation.

In this example, the guide 141 comprises a split (not shown) which runs down the length of the guide 141. The guide 141 also comprises an array of closely spaced ribs 143 which, in this case, are formed integrally to the guide 141. The guide 141 therefore has a generally corrugated appearance.

As shown in FIG. 7B, when a guide 141 is to be applied to the colonoscope 131, the guide 141 is fitted around an upper end of the arcuate portion 135 (e.g. an end distal from the colonoscope 131) of the applicator 133. As shown in FIG. 7C, the guide 141 is fed down the arcuate portion 135 towards the colonoscope 131. The arcuate portion tapers away from the colonoscope 131, such that as the guide 141 is fed over the arcuate portion, the resilient deformation of the guide 141 results in the circumferential width of the split increasing so as to allow the guide 141 to be inserted over the colonoscope 131. The restorative force from this deformation acts to urge the sides of the split towards one another, thereby reducing the circumferential width of the split and closing (or partially closing) the split behind the colonoscope 131 once the guide 141 has been inserted over the colonoscope 131 (see FIG. 7D). The restorative force of the guide 141 also helps to secure the guide 141 around the colonoscope 131.

In some embodiments, the applicator 133 may further comprise a guide closing portion arranged to urge the sides of the split towards one another to close or partially close the split (in combination with or in place of restorative force from deformation of the guide 141). The applicator 133 may also have an attachment portion such as a clamp for attaching the applicator 133 to the colonoscope 131. This may allow the surgeon or clinician to manipulate the colonoscope 131 via movement of the applicator 133, and/or may allow the applicator 133 to be supported by the colonoscope 131 when the guide 141 is being applied thereto (for instance the surgeon or clinician may hold the colonoscope 131 still with one hand via the applicator 133, and thread the guide 141 onto and along the colonoscope 131 with the other hand). The attachment portion may be part of or all of the colonoscope-engaging section 137. In embodiments, the attachment portion may have a trigger mechanism which allows the attachment portion or clamp to be deployed by the surgeon or clinician once the applicator 133 is in a suitable portion on the colonoscope 131.

The applicator 133 may be mountable to the colonoscope 131 (for instance with an attachment portion, as discussed above), in a manner which allows part of the applicator 133 (preferably including the handle of the applicator 133) to rotate about the longitudinal axis of the colonoscope 131.

As described above, the guide 141 of FIG. 7 has a generally corrugated appearance. FIG. 8A depicts such a corrugated guide 145. The guide 145 has a tubular structure and comprises corrugations (or ribs) 147 along its length. The presence of such corrugations (or ribs) 147 may enable the guide to traverse the tight bends of the colon more easily, without being removed from a colonoscope. As depicted in FIG. 8B, a guide according to the invention may comprise fenestrations or apertures 149 instead of (or indeed in addition to) the corrugations 147 shown in FIG. 8A.

Referring now to FIG. 9A-D there is depicted a method of removing a polyp according to an embodiment of the present invention. During a colonoscopy, a colonoscope 151 is passed through the rectum 153 into the colon 155. The colonoscope 151 is advanced along the colon 155 until a polyp 157 is located. If the polyp 157 is a small polyp, for example less than around 7 mm, it may be excised and withdrawn through a port (not shown) in the colonoscope 151. However, if the polyp 157 is large, a guide 159, in this case a guide having a split along its length, is mounted to the colonoscope 151 and advanced along the length of the colonoscope 151 until the guide 159 reaches the site of the polyp 157 (see FIG. 9B). The guide 159 is generally tubular in shape and, in use, generally surrounds the colonoscope 151 to form a sheath. The guide 159 has a channel 161 along its length through which a surgical instrument 163 or polyp can be passed.

In this case, an excision instrument 163 is advanced along the channel 161 until it reaches the site of the polyp 157. The polyp 157 is then excised and captured in a Roth net attached to the instrument 163. In this example, the surgical instrument 163, polyp 157 and guide 159 are removed from the colon 155 simultaneously while the colonoscope 151 remains in place (see FIG. 9D). However, as will be appreciated, in some other embodiments the polyp 157 and surgical instrument 163 could be removed from the colon 155 through the channel 161 of the guide 159. Whichever approach is taken, the colonoscope 151 can remain in place while the polyp 157 is withdrawn from the colon 155.

If a further large polyp 157 is identified, the guide 159 can once again be mounted to the colonoscope 151 and advanced along the length of the colonoscope 151. Excision and withdrawal of the polyp 157 can then be performed as described above. In this way, multiple large polyps can be removed from the colon 155 without having to remove and re-insert the colonoscope 155 which can cause damage to the lining of the colon 155 and/or discomfort for the patient. The presence of the colonoscope 151 initially inserted into the colon acts as a “track” for the guide 159 to slide along. The presence of the guide 159 then allows repeated removal and insertion of the colonoscope 151 from the colon, if required, without repeated complex navigation of the colon.

Referring now to FIG. 10 there is depicted a guide and method of removing a polyp according to an embodiment of the present invention. Unlike the guide depicted in FIG. 9, the guide 171 of FIG. 10 does not have a split along its length. The guide 171, which is tubular in this instance, is mounted to the colonoscope 173 prior to insertion of the colonoscope 173 into the colon 175 (see FIG. 10A). Corresponding attachment features (in this case ribs 177 and 179) on the guide 171 and colonoscope 173 are used to attach the colonoscope 173 and guide 171 together (in this case at this distal end of the colonoscope) for insertion into the colon 175. Such attachment prevents axial compression of the guide 171 on insertion into the colon 175 and allows the guide 171 to progress along the colon 175 with the colonoscope 173.

Once the colonoscope 173 and guide 171 are attached to one another, they are inserted into the colon 175 via the rectum 181 (FIG. 10B part 1). An excision instrument is passed through a port in the colonoscope until it reaches the location of the polyp. The polyp is then excised from the colon 175 and captured in a Roth net, for example. At this stage, the attachment features between the colonoscope 173 and guide 171 are disengaged remotely, such that the colonoscope 173 can be removed from the colon 175 with the excised polyp, leaving the guide 171 in place. The colonoscope 173 can then be re-inserted into the colon 175 via the guide 171 which acts as a lining between the wall of the colon and the colonoscope 173, thereby minimising the likelihood of damage to the lining of the colon 175 and/or patient discomfort. It will be appreciated that although the guide 171 of FIG. 10 does not comprise a split along its length, a similar method could be achieved using a guide having a split along its length.

It will be appreciated that numerous modifications to the above described guide and method may be made without departing from the scope of the invention as defined in the appended claims. As one example, in an alternative embodiment the applicator may have a straight portion which expands the guide to fit it onto the colonoscope rather than an arcuate portion. Moreover, any one or more of the above described preferred embodiments could be combined with one or more of the other preferred embodiments to suit a particular application.

Optional and/or preferred features may be used in other combinations beyond those described herein and optional and/or preferred features described in relation to one aspect of the invention may also be present in another aspect of the invention, where appropriate.

The described and illustrated embodiments are to be considered as illustrative and not restrictive in character, it being understood that only the preferred embodiments have been shown and described and that all changes and modifications that come within the scope of the inventions as defined in the claims are desired to be protected. It should be understood that while the use of words such as “preferable”, “preferably”, “preferred” or “more preferred” in the description suggest that a feature so described may be desirable, it may nevertheless not be necessary and embodiments lacking such a feature may be contemplated as within the scope of the invention as defined in the appended claims. In relation to the claims, it is intended that when words such as “a,” “an,” or “at least one,” are used to preface a feature there is no intention to limit the claim to only one such feature unless specifically stated to the contrary in the claim.

1-29. (canceled)

30. An instrument guide for use with a colonoscope, the guide being adapted to be mounted to the colonoscope; and wherein the guide defines a channel along which an instrument can be passed during use.

31. The guide according to claim 30 wherein the channel is configured such that the instrument can be passed generally alongside the colonoscope when the guide is mounted to the colonoscope.

32. The guide according to claim 30 wherein the guide comprises a longitudinal array of generally annular or C-shaped ribs.

33. The guide according to claim 30 wherein the guide is generally in the form of a hollow tubular structure.

34. The guide according to claim 33 wherein the tubular structure is resiliently deformable.

35. The guide according to claim 33 wherein the tubular structure comprises at least one of corrugations and fenestrations.

36. The guide according to claim 30 wherein the guide is deformable in a radial direction.

37. The guide according to claim 30 further comprising a stiffening element positioned to stiffen the guide in a longitudinal direction.

38. The guide according to claim 30 wherein the guide is fabricated from a thermoplastic material.

39. The guide according to claim 30 wherein the guide further comprises an attachment feature for coupling of the guide to the colonoscope.

40. The guide according to claim 39 wherein the attachment feature is a remotely actuable coupling mechanism.

41. The guide according to claim 30 wherein the instrument is the colonoscope.

42. A kit of parts comprising a colonoscope and a guide according to claim 30.

43. A method of removing a polyp from a colon, the method comprising:
inserting a colonoscope into the colon;
inserting a guide according to claim 30 into the colon;
mounting the guide to the colonoscope;
inserting an excision instrument for excising a polyp from a wall of the colon;
excising the polyp using said excision instrument; and
withdrawing the excised polyp from the colon.

44. The method of claim 43 further comprising inserting a withdrawal instrument prior to the step of withdrawing the excised polyp from the colon, wherein the step of withdrawing the excised polyp from the colon is achieved using the withdrawal instrument.

45. The method of claim 43 wherein the step of inserting a colonoscope into the colon takes place prior to the step of inserting the guide into the colon.

46. The method of claim 43 wherein the step of mounting the guide to the colonoscope comprises connecting the guide to the colonoscope.

47. The method of claim 43 wherein the steps of inserting the colonoscope into the colon and inserting the guide into the colon are performed substantially simultaneously.

48. The method of claim 46 wherein the step of connecting the guide to the colonoscope takes place prior to the steps of inserting the colonoscope into the colon and inserting the guide into the colon.

49. The method according to claim 43 wherein the method is used to remove multiple polyps from the colon and following the step of withdrawing the excised polyp from the colon the method further comprises:
excising a further polyp using said excision instrument; and
withdrawing the excised further polyp from the colon, optionally using a withdrawal instrument.
